(19) United States
(12) Patent Application Publication
(10) Pub. No.: US 2017/0315135 A1
(43) Pub. Date: Nov. 2, 2017

(54) BIOMARKERS USEFUL IN THE TREATMENT OF SUBJECTS HAVING RETINITS PIGMENTOSA

(71) Applicant: THE JOHNS HOPKINS UNIVERSITY, Baltimore, MD (US)

(72) Inventors: Peter A. Campochiaro, Baltimore, MD (US); Lili Lu, Joppa, MD (US)

(21) Appl. No.: 15/525,958

(22) PCT Filed: Nov. 11, 2015
(86) PCT No.: PCT/US2015/060089

§ 371 Date: May 11, 2017

Related U.S. Application Data
(60) Provisional application No. 62/078,138, filed on Nov. 11, 2014.

(51) Int. Cl. G01N 33/68

(52) U.S. Cl. CPC G01N 33/6893; G01N 2800/56; G01N 2800/164; G01N 2800/52

(57) ABSTRACT

The present invention provides biomarkers of oxidative stress in subjects with Retinitis Pigmentosa and their use in identifying subjects in need of treatment and methods for staging the severity of the disease.

### REFERENCE TO RELATED APPLICATIONS

This application claims the benefit U.S. Provisional Patent Application No. 62/078,138, filed on Nov. 11, 2014, which is hereby incorporated by reference for all purposes as if fully set forth herein.

### BACKGROUND OF THE INVENTION

Retinitis Pigmentosa (RP) is the term used for a genetically heterogenous group of inherited retinal degenerations. Findings may be limited to the eyes or the eye findings may be part of a syndrome the most common of which is Usher's Syndrome in which deafness accompanies the retinal disease. In each disorder the inciting event is a mutation that leads to the death of rod photoreceptors, initially causing night blindness. Rods are the major consumers of oxygen in the retina and the loss of rods causes an increase in the tissue oxygen level in the outer retina. This activates NADPH oxidase causing accumulation of superoxide radicals in the cytosol and also increases their generation in mitochondria of cones. The excess superoxide radicals overwhelm superoxide dismutase 1 (SOD1) and SOD2 and cause a chain reaction by which other free radicals are generated including some that are even more damaging than superoxide radicals, such as hydroxyl radicals and peroxynitrite. The free radicals attack proteins, lipids, and DNA causing specific modifications that indicate that oxidative damage has occurred. Oxidative damage to lipids results in lipid hydroperoxides that break down to form 4-hydroxynonenal, malondialdehyde (MDA), and acrolein. The most common modification to proteins from oxidative damage is the formation of carbonyl adducts. These modifications can impair the function of macromolecules and while there are endogenous repair processes, they are overwhelmed by severe oxidative stress resulting in reduced cellular function and eventually apoptosis. After rods are eliminated from the photoreceptor layer, oxidative stress in the outer retina is severe and leads to gradual cone cell death usually starting in the midperiphery where cone density is low and then spreading peripherally and posteriorly. The posterior spread of cone death results in constriction of the visual field and eventually a central island of vision and its elimination causes blindness.

Clinical signs of RP include pigmentary changes in the retina, often around blood vessels and characterized as “bone spicule-like pigmentation”, constriction of retinal vessels, and optic disc pallor. Spectral domain optical coherence tomography can show thinning of the retina in areas of photoreceptor cell loss and with segmentation the loss is seen in the outer nuclear layer. Visual field testing shows constriction of the visual fields and electroretinograms show reduced a- and b-wave amplitudes.

Currently, there is no approved therapy that stops the evolution of the disease or restores vision. The therapeutic approach is restricted to slowing down the degenerative process by sunlight protection and vitamin A supplementation, treating complications (cataract and macular edema), and helping patients to cope with the social and psychological impact of blindness. Although the Argis II Retinal Prosthesis System was approved by FDA in 2013 as an implanted device to treat adults with severe RP, it only produces the sensation of light, thereby helping patients identify the location or movement of objects and people; the device is not disease modifying.

In order to test new treatments, it is necessary to have robust measures of disease progression. The most widely accepted functional measure of disease progression is loss of visual field; however, there are several ways to assess visual fields and their value may differ at different stages of the disease. Goldman visual fields provide a good assessment of peripheral visual fields and are useful during the early stages of cone cell loss. Assessment of retinal function by full field ERGs is also most useful in early stage disease because signals are low and often unrecordable in later stage disease. Automated measures of retinal sensitivity provide measurements on posterior retina which is normal early in the disease, but in later stage disease is more sensitive and quantitative than Goldman visual fields. Determining the annual rate of change in the width of the central area of intact inner segment ellipsoid zone by spectral domain OCT provides a fairly sensitive anatomic measure of the disease progression in relatively advanced disease. A problem shared by all of these outcome measures is that their rate of change over time is low necessitating long term clinical trials to have a chance of detecting treatment effects.

As such, the identification and development of biomarkers that could serve as interim readouts of drug activity, bioavailability, and compliance would be a major benefit for planning and carrying out clinical trials.

### SUMMARY OF THE INVENTION

The present inventors have determined that the ongoing oxidative stress in patients with RP causes oxidative damage to secreted macromolecules as well as those that are retained within cells. Therefore novel markers of oxidative stress should be detectable in aqueous humor and serum of patients with RP and should be greater than the baseline levels in aqueous humor and serum from normal control patients. Furthermore, as cone death progresses, oxidative stress should increase and therefore the level of any particular marker of oxidative damage is likely to increase in an RP patient over time, and in a population of RP patients there is a positive correlation between level of an oxidative damage marker and stage of disease.

Thus, in one aspect, the present invention provides methods for assessing the severity or stage of RP in a patient diagnosed with the disease by measuring markers which indicate increasing or decreasing oxidative stress in the aqueous humor and serum of patients. The methods disclosed herein can also be used to monitor the progression of the disease in a patient over time and determine whether a course of treatment is effective in slowing or stopping the progression of the disease in the eye.

In accordance with an embodiment, the present invention provides a method for quantifying or staging the severity of disease in a subject diagnosed as having Retinitis Pigmentosa comprising: a) obtaining a biological sample from the subject; b) providing a control biological sample; c) measuring one or more of the following: cupric reducing antioxidant capacity, GSH/GSSG ratio, and carbonyl adduct level in the sample of a) and b); d) comparing the one or more measurements of c) in the sample from the subject to the control sample; and e) quantifying or staging the severity of disease in the subject as progressing in stage, or increasing in severity, if the levels of cupric reducing antioxidant capacity and/or GSH/GSSG ratio is reduced relative to the control sample, and/or if the levels of carbonyl adduct level is elevated relative to the control sample.

Therefore, in accordance with another embodiment, the present invention provides a method for treating a subject having Retinitis Pigmentosa comprising: a) obtaining a biological sample from the subject; b) providing a control biological sample; c) measuring one or more of the following: cupric reducing antioxidant capacity, GSH/GSSG ratio, carbonyl adduct level in the sample of a) and b); d) comparing the one or more measurements of c) in the sample from the subject to the control sample; e) quantifying or staging the severity of disease in the subject as progressing in stage, or increasing in severity, if the levels of cupric reducing antioxidant capacity and/or GSH/GSSG ratio is reduced relative to the control sample, and/or if the levels of carbonyl adduct level is elevated relative to the control sample; and f) selecting a course of treatment for the disease in the subject which is based on the stage or severity of disease indicated in e).

### BRIEF DESCRIPTION OF THE DRAWINGS

FIG. 1 illustrates the comparison of protein carbonyl content on proteins in aqueous samples from patients with retinitis pigmentosa (RP) and controls. Each bar represents the mean (±standard deviation) protein carbonyl content per mg protein and statistical comparison was made by Student's unpaired t-test.

FIG. 2 depicts the comparison of the ratio of reduced glutathione to oxidized glutathione (GSH/GSSG) in aqueous samples from patients with retinitis pigmentosa (RP) and controls. The bars represent the mean (±standard deviation) GSH/GSSH ratio and statistical comparison was made by unpaired t-test.

### DETAILED DESCRIPTION OF THE INVENTION

Tissues combat oxidative stress through the endogenous antioxidant defense system which has several components. Individuals vary in the effectiveness of their antioxidant defense system based upon their genetic makeup. Patients also vary with regard to antioxidants in their diet. Patients with RP who have a particularly effective antioxidant defense system and/or an antioxidant-rich diet should have a slower rate of cone cell loss and hence progression of disease. This variability in disease progression adds “noise” to attempts to assess the impact of a treatment on disease progression. Assessment of the reducing power, also known as total antioxidant capacity (TAC) of a tissues provides a readout of the endogenous antioxidant defense system, the level of exogenous antioxidants in the tissue, and the current level of oxidative stress (because antioxidant capacity is reduced by oxidative stress). It can therefore provide an assessment of how well the individual is coping with the current load of oxidative stress in the tissue. The addition of an antioxidant therapy should increase the TAC in the target tissue if it has a chance of reducing oxidative damage in the tissue. Thus, in accordance with some embodiments, increase in TAC above baseline can provide assessments of compliance and bioavailability, and serve as a biomarker to predict therapeutic effect.

The ferric reducing ability of plasma (FRAP) assay is advantageous because it is relatively inexpensive, and convenient (Anal. Biochem. 1996;239:70-6). Measurement of TAC using FRAP decreases with aging and at any age, there is correlation between antioxidant capacity and oxidative damage in various tissues (Rejuvenation Res. 2006;9:470-4; Free Radic. Biol. Med. 2002;33:597-604; Ann. Biol. Clin. 2001;59:453-9). The cupric reducing antioxidant capacity (CUPRAC) method is similar to FRAP, but has better kinetics and is more reproducible (Free Radic. Res. 2005;39:949-61; Mol. Cell Biochem. 2009;323:139-42). Reduced glutathione (GSH) is a major intracellular non-protein —SH compound and is the most important intracellular hydrophilic antioxidant. Under oxidative conditions, GSH is reversibly oxidized to glutathione disulfide (GSSG) and under reducing conditions, GSH is regenerated. Thus the GSH/GSSG ratio provides a measure of antioxidant status similar to antioxidant capacity (Rejuvenation Res. 2006;9:169-81).

Thus, in accordance with one or more embodiments, the present invention provides biomarkers, such as CUPRAC and GSH/GSSH ratio, which are shown to be reduced in the aqueous humor with RP and the level of reduction correlates with the increased stage of cone cell loss.

In accordance with an embodiment, the present invention provides a method for quantifying or staging the severity of disease in a subject diagnosed as having Retinitis Pigmentosa comprising: a) obtaining a biological sample from the subject; b) providing a control biological sample; c) measuring one or more of the following: cupric reducing antioxidant capacity, GSH/GSSG ratio, and carbonyl adduct level in the sample of a) and b); d) comparing the one or more measurements of c) in the sample from the subject to the control sample; and e) quantifying or staging the severity of disease in the subject as progressing in stage, or increasing in severity, if the levels of cupric reducing antioxidant capacity and/or GSH/GSSG ratio is reduced relative to the control sample, and/or if the levels of carbonyl adduct level is elevated relative to the control sample.

Therefore, in accordance with another embodiment, the present invention provides a method for treating a subject having Retinitis Pigmentosa comprising: a) obtaining a biological sample from the subject; b) providing a control biological sample; c) measuring one or more of the following: cupric reducing antioxidant capacity, GSH/GSSG ratio, carbonyl adduct level in the sample of a) and b); d) comparing the one or more measurements of c) in the sample from the subject to the control sample; e) quantifying or staging the severity of disease in the subject as progressing in stage, or increasing in severity, if the levels of cupric reducing antioxidant capacity and/or GSH/GSSG ratio is reduced relative to the control sample, and/or if the levels of carbonyl adduct level is elevated relative to the control sample; and f) selecting a course of treatment for the disease in the subject which is based on the stage or severity of disease indicated in e).

In a further embodiment, the present invention provides a method for monitoring the treatment of a subject having Retinitis Pigmentosa comprising: a) obtaining a biological sample from the subject; b) providing a control biological sample; c) measuring one or more of the following: cupric reducing antioxidant capacity, GSH/GSSG ratio, carbonyl adduct level in the sample of a) and b); d) comparing the one or more measurements of c) in the sample from the subject to the control sample; e) determining the stage or the severity of disease in the subject, wherein if the levels of cupric reducing antioxidant capacity and/or GSH/GSSG ratio is reduced relative to the control sample, and/or if the levels of carbonyl adduct level is elevated relative to the control sample then the disease is identified as progressing in stage, or increasing in severity, or if the levels of cupric reducing antioxidant capacity and/or GSH/GSSG ratio is elevated relative to the control sample and/or if the levels of carbonyl adduct level is reduced relative to the control sample then the disease is identified as reducing in stage, or decreasing in severity; f) selecting a course of treatment for the disease in the subject which is based on the stage or severity of disease indicated in e); and g) after administration of the course of treatment to the subject, repeating steps a)-f) one or more times.

In accordance with one or more embodiments, the present inventors have determined that because oxidative stress is the primary insult to cones in the eye, the amount of oxidative damage measured on macromolecules in the aqueous humor will correlate with ongoing oxidative damage to cones, and therefore these provide a measure of disease activity in the eye of a subject with RP. As such, the importance of these inventive markers is that at any stage of disease, the clinician's treatment objective is to reduce disease activity and hence ongoing damage. Therefore the present invention allows one of skill in the art to determine if a particular treatment is having the desired effect, in a much shorter time-frame, without waiting to see if it slows the loss of cone function in the subject.

For example, after selecting a cohort of patients having RP, one of skill in the art would measure one or more of the following markers: cupric reducing antioxidant capacity, GSH/GSSG ratio, and carbonyl adduct level in samples from the subject before treatment as an indicator of baseline activity. The treatment would then begin with n-acetylcystine amide, or another suitable treatment, and then further samples would be taken at various time points during therapy. If the markers show a decrease in oxidative stress, that will correlate with a decrease in disease activity. Then over time, the reduction in disease activity markers should correlate with reduction in rate of loss of cone function in the subject.

In another aspect, use of the cupric reducing antioxidant capacity, GSH/GSSG ratio, and carbonyl adduct level in samples from the subject can be used to screen for other potential drugs which can reduce oxidative stress in the eye.

An “agent” is understood herein to include a therapeutically active compound or a potentially therapeutic active compound, e.g., an antioxidant. An agent can be a previously known or unknown compound. As used herein, an agent is typically a non-cell based compound, however, an agent can include a biological therapeutic agent, e.g., peptide or nucleic acid therapeutic, e.g., siRNA, shRNA, cytokine, antibody, etc.

As used herein “amelioration” or “treatment” is understood as meaning to lessen or decrease at least one sign, symptom, indication, or effect of a specific disease or condition. For example, amelioration or treatment of retinitis pigmentosa (RP) can be to reduce, delay, or eliminate one or more signs or symptoms of RP including, but not limited to, a reduction in night vision, a reduction in overall visual acuity, a reduction in visual field, a reduction in the cone density in one or more quadrants of the retina, thinning of retina, particularly the outer nuclear layer, reduction in a- or b-wave amplitudes on scotopic or photopic electroretinograms (ERGs); or any other clinically acceptable indicators of disease state or progression. Amelioration and treatment can require the administration of more than one dose of an agent, either alone or in conjunction with other therapeutic agents and interventions. Amelioration or treatment does not require that the disease or condition be cured.

“Antioxidant” as used herein is understood as a molecule capable of slowing or preventing the oxidation of other molecules. Oxidation is a chemical reaction that transfers electrons from a substance to an oxidizing agent. Such reactions can be promoted by or produce superoxide anions or peroxides. Oxidation reactions can produce free radicals, which start chain reactions that damage cells. Antioxidants terminate these chain reactions by removing free radical intermediates, and inhibit other oxidation reactions by being oxidized themselves. As a result, antioxidants are often reducing agents such as thiols, ascorbic acid or polyphenols. Antioxidants include, but are not limited to, α-tocopherol, ascorbic acid, Mn(III)tetrakis (4-benzoic acid) porphyrin, α-lipoic acid, and n-acetylcysteine.

“Co-administration” as used herein is understood as administration of one or more agents to a subject such that the agents are present and active in the subject at the same time. Co-administration does not require a preparation of an admixture of the agents or simultaneous administration of the agents.

The terms “effective amount,” or “effective dose” refers to that amount of an agent to produce the intended pharmacological, therapeutic or preventive result. The pharmacologically effective amount results in the amelioration of one or more signs or symptoms of a disease or condition or the advancement of a disease or condition, or causes the regression of the disease or condition. For example, a therapeutically effective amount preferably refers to the amount of a therapeutic agent that decreases the loss of night vision, the loss of overall visual acuity, the loss of visual field, by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or more as compared to an untreated control subject over a defined period of time, e.g., 2 weeks, one month, 2 months, 3 months, 6 months, one year, 2 years, 5 years, or longer. More than one dose may be required to provide an effective dose.

As used herein, the terms “effective” and “effectiveness” includes both pharmacological effectiveness and physiological safety. Pharmacological effectiveness refers to the ability of the treatment to result in a desired biological effect in the patient. Physiological safety refers to the level of toxicity, or other adverse physiological effects at the cellular, organ and/or organism level (often referred to as side-effects) resulting from administration of the treatment. On the other hand, the term “ineffective” indicates that a treatment does not provide sufficient pharmacological effect to be therapeutically useful, even in the absence of deleterious effects, at least in the unstratified population. (Such a treatment may be ineffective in a subgroup that can be identified by the expression profile or profiles.) “Less effective” means that the treatment results in a therapeutically significant lower level of pharmacological effectiveness and/or a therapeutically greater level of adverse physiological effects, e.g., greater liver toxicity.

Thus, in connection with the administration of a drug, a drug which is “effective against” a disease or condition indicates that administration in a clinically appropriate manner results in a beneficial effect for at least a statistically significant fraction of patients, such as a improvement of symptoms, a cure, a reduction in disease signs or symptoms, extension of life, improvement in quality of life, or other effect generally recognized as positive by medical doctors familiar with treating the particular type of disease or condition.

“Oxidative stress related ocular disorders” as used herein include, but are not limited to, retinitis pigmentosa, macular degeneration including age related macular degeneration (AMD) both wet and dry, diabetic retinopathy, Lebers optic neuropathy, and optic neuritis.

“Peroxidases” or “a peroxide metabolizing enzyme” are a large family of enzymes that typically catalyze a reaction of the form:

ROOR¹+electron donor (2e−)+2H+→ROH+R¹OH.

For many of these enzymes the optimal substrate is hydrogen peroxide, wherein each R is H, but others are more active with organic hydroperoxides such as lipid peroxides. Peroxidases can contain a heme cofactor in their active sites, or redox-active cysteine or selenocysteine residues.

The phrase “pharmaceutically acceptable carrier” is art recognized and includes a pharmaceutically acceptable material, composition or vehicle, suitable for administering compounds of the present invention to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. For example, pharmaceutically acceptable carriers for administration of cells typically is a carrier acceptable for delivery by injection, and do not include agents such as detergents or other compounds that could damage the cells to be delivered. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations, particularly phosphate buffered saline solutions which are preferred for intraocular delivery.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, α-tocopherol, and the like; and metal chelating agents, such as citric acid, ethylenediamine tetraacetic acid (EDTA), sorbitol, tartaric acid, phosphoric acid, and the like.

Formulations of the present invention include those suitable for oral, nasal, topical, transdermal, buccal, sublingual, intramuscular, intraperotineal, intraocular, intravitreal, subretinal, and/or other routes of parenteral administration. The specific route of administration will depend, inter alia, on the specific cell to be targeted. The formulations may conveniently be presented in unit dosage form and may be prepared by any methods well known in the art of pharmacy. The amount of active ingredient that can be combined with a carrier material to produce a single dosage form will generally be that amount of the compound that produces a therapeutic effect.

As used herein, “plurality” is understood to mean more than one. For example, a plurality refers to at least two, three, four, five, or more.

As used herein, “prevention” is understood as to limit, reduce the rate or degree of onset, or inhibit the development of at least one sign or symptom of a disease or condition particularly in a subject prone to developing the disease or disorder. For example, a subject having a mutation in a gene, such as the opsin gene, is likely to develop RP. The age of onset of one or more symptoms of the disease can sometimes be determined by the specific mutation. Prevention can include the delay of onset of one or more signs or symptoms of RP and need not be prevention of appearance of at least one sign or symptom of the disease throughout the lifetime of the subject. Prevention can require the administration of more than one dose of an agent or therapeutic.

“Small molecule” as used herein is understood as a compound, typically an organic compound, having a molecular weight of no more than about 1500 Da, 1000 Da, 750 Da, or 500 Da. In an embodiment, a small molecule does not include a polypeptide or nucleic acid including only natural amino acids and/or nucleotides.

A “subject” as used herein refers to living organisms. In certain embodiments, the living organism is an animal, in certain preferred embodiments, the subject is a mammal, in certain embodiments, the subject is a domesticated mammal or a primate including a non-human primate. Examples of subjects include humans, monkeys, dogs, cats, mice, rats, cows, horses, goats, and sheep. A human subject may also be referred to as a patient.

A subject “suffering from or suspected of suffering from” a specific disease, condition, or syndrome has a sufficient number of risk factors or presents with a sufficient number or combination of signs or symptoms of the disease, condition, or syndrome such that a competent individual would diagnose or suspect that the subject was suffering from the disease, condition, or syndrome. Methods for identification of subjects suffering from or suspected of suffering from conditions such as RP and age-related macular degeneration (AMD) is within the ability of those in the art. Subjects suffering from, and suspected of suffering from, a specific disease, condition, or syndrome are not necessarily two distinct groups.

As used herein, “superoxide dismutase” is understood as an enzyme that catalyzes the dismutation of superoxide radical (O₂⁻) into either oxygen (O₂⁻) or hydrogen peroxide (H₂O₂). Examples include, but are not limited to SOD1, SOD2, and SOD3. SOD1 and SOD3 are two isoforms of Cu—Zn-containing superoxide dismutase enzymes exist in mammals. Cu—Zn-SOD or SOD1, is found in the intracellular space, and extracellular SOD (ECSOD or SOD3) predominantly is found in the extracellular matrix of most tissues.

“Therapeutically effective amount,” as used herein refers to an amount of an agent which is effective, upon single or multiple dose administration to the cell or subject, in prolonging the survivability of the patient with such a disorder, reducing one or more signs or symptoms of the disorder, preventing or delaying and the like beyond that expected in the absence of such treatment.

An agent or other therapeutic intervention can be administered to a subject, either alone or in combination with one or more additional therapeutic agents or interventions, as a pharmaceutical composition in mixture with conventional excipient, e.g., pharmaceutically acceptable carrier, or therapeutic treatments.

The pharmaceutical agents may be conveniently administered in unit dosage form and may be prepared by any of the methods well known in the pharmaceutical arts, e.g., as described in Remington's Pharmaceutical Sciences (Mack Pub. Co., Easton, Pa., 1990). Formulations for parenteral administration may contain as common excipients such as sterile water or saline, polyalkylene glycols such as polyethylene glycol, oils of vegetable origin, hydrogenated naphthalenes and the like. In particular, biocompatible, biodegradable lactide polymer, lactide/glycolide copolymer, or polyoxyethylene-polyoxypropylene copolymers may be useful excipients to control the release of certain agents.

In some embodiments, the treatment of a subject identified as having RP can include antioxidant therapy, such as N-acetyl cysteine (NAC), N-acetyl cysteine amide (NACA), ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, α-tocopherol, and the like; and metal chelating agents, such as citric acid, ethylenediamine tetraacetic acid (EDTA), sorbitol, tartaric acid, phosphoric acid, and the like.

In other embodiments, the treatment of a subject identified as having RP can include administering an isolated polynucleotide encoding human glutamate cysteine ligase and human glutathione synthase in an expression construct as taught in International Application No. PCT/US2013/076433, and incorporated by reference herein in its entirety.

In some embodiments, the one or more antioxidant agents is administered intraocularly, subretinally, intravitreally, orally, intravenously, intramuscularly, intramedullarily, intrathecally, intraventricularly, transdermaly, subcutaneously, intraperitoneally, intranasally, enterally, topically, sublingually, or rectally.

It will be appreciated that the actual preferred amounts of active compounds or agents used in a given therapy will vary according to e.g., the specific compound being utilized, the particular composition formulated, the mode of administration, characteristics of the subject, e.g., the species, sex, weight, general health and age of the subject. Optimal administration rates for a given protocol of administration can be readily ascertained by those skilled in the art using conventional dosage determination tests conducted with regard to the foregoing guidelines.

The agents can, for example, be administered by injection, intraocularly, intravitreally, subretinal, intravenously, intraarterially, subdermally, intraperitoneally, intramuscularly, or subcutaneously; or orally, buccally, nasally, transmucosally, directly to a diseases organ by catheter, topically, or in an ophthalmic preparation, with a dosage ranging from about 0.001 to about 100 mg/kg of body weight, or according to the requirements of the particular drug and more preferably from 0.5-10 mg/kg of body weight. It is understood that when a compound is delivered directly to the eye, considerations such as body weight have less bearing on the dose.

Frequency of dosing will depend on the agent administered, the progression of the disease or condition in the subject, and other considerations known to those of skill in the art. For example, pharmacokinetic and pharmacodynamic considerations for compositions delivered to the eye, or even compartments within the eye, are different, e.g., clearance in the subretinal space is very low. Therefore, dosing can be as infrequent as once a month, once every three months, once every six months, once a year, once every five years, or less. If systemic administration of antioxidants is to be performed in conjunction with administration of expression constructs to the subretinal space, it is expected that the dosing frequency of the antioxidant will be higher than the expression construct, e.g., one or more times daily, one or more times weekly.

Dosing may be determined in conjunction with monitoring of one or more signs or symptoms of the disease, e.g., visual acuity, visual field, night visions, etc. The amount of active ingredient that may be combined with the carrier materials to produce a single dosage form will vary depending upon the host treated and the particular mode of administration. A typical preparation will contain from about 1% to about 95% active compound (w/w). Alternatively, such preparations contain from about 20% to about 80% active compound. Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

In accordance with some embodiments, the methods taught herein are useful for managing the dosage of various RP treatments, as well as determining whether the treatments are having the effect of lowering the oxidative products in the eye, well before any physical measurements of vision are manifested. Thus, the methods of the present invention can be used in conjunction with various RP treatments to better manage the disease.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, TWEEN® 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as TWEENs® or SPANs® and/or other similar emulsifying agents or bioavailability enhancers which are commonly used in the manufacture of pharmaceutically acceptable solid, liquid, or other dosage forms may also be used for the purposes of formulation.

As used herein, “susceptible to” or “prone to” or “predisposed to” a specific disease or condition and the like refers to an individual who based on genetic, environmental, health, and/or other risk factors is more likely to develop a disease or condition than the general population. An increase in likelihood of developing a disease may be an increase of about 10%, 20%, 50%, 100%, 150%, 200%, or more.

Aqueous and serum MDA, carbonyl content, CUPRAC, and GSH/GSSH will be measured in RP patients and controls. Serum bilirubin is measured in RP patients and controls.

It will be understood by those of skill in the art, that the methods for assessing Aqueous and serum MDA, carbonyl content, CUPRAC, and GSH/GSSH will be measured in RP patients and controls are not limited to any particular assay or method, and can use any known analytical method.

### EXAMPLES

Clinical studies have enrolled patients with RP and controls (patients who will be undergoing surgery for macular hole, epiretinal membrane, or vitreomacular traction). Patients with RP who provide informed consent will have an anterior chamber tap and blood draw at the end of the first routine clinic follow up visit after having signed the consent form. Blood draw and anterior chamber tap will be repeated at 6 month intervals for a total of 5 times over the course of 2 years. Control patients who provide informed consent will have an anterior chamber tap and blood draw at the onset of their vitreoretinal surgery in the operating room. Age, gender, smoking history, alcohol intake, open angle glaucoma status and current use of supplements will be recorded for all RP patients and controls. Aqueous and serum samples will be stored at −80° C. until measurement of MDA, carbonyl content, CUPRAC, and GSH/GSSH. Bilirubin level will also be measured in serum.

Anterior chamber tap in the clinic in patients with RP.

The patient will be seated at a slit lamp and a drop of topical anesthetic will be placed in the study eye. A 30 gauge needle is passed through the limbus into the anterior chamber and 0.1 ml of aqueous humor is removed. There are no nerves at the limbus below the epithelium and therefore the topical anesthetic provides complete anesthesia and there is no pain associated with the procedure. Anterior chamber tap is a routine procedure that is extremely safe. It is done to deal with severe increased intraocular pressure after intravitreous injections, particularly injection of gas during pneumatic retinopexy. Anterior chamber taps have been done as part of several prior studies and there have been no related complications or adverse events.

### Anterior Chamber Tap in the Operating Room in Controls.

After retrobulbar anesthesia and after the patient is prepped and draped, a 30 gauge needle is inserted into the anterior chamber and 100 μl of aqueous is aspirated and the sample will be put on ice until frozen. The surgery is then done according to standard care.

### Peripheral Blood Draw.

This procedure is commonplace and there are no risks to exposure to transmissible diseases if ones blood is being drawn using aseptic conditions. A patient may feel light headed or faint, or experience pain or discomfort at the site of puncture during the procedure. There may be possible bruising or swelling at the puncture site, rarely there may be an infection.

### Example 1

Comparison of protein carbonyl content on proteins in aqueous humor samples from patients with retinitis pigmentosa (RP) and controls.

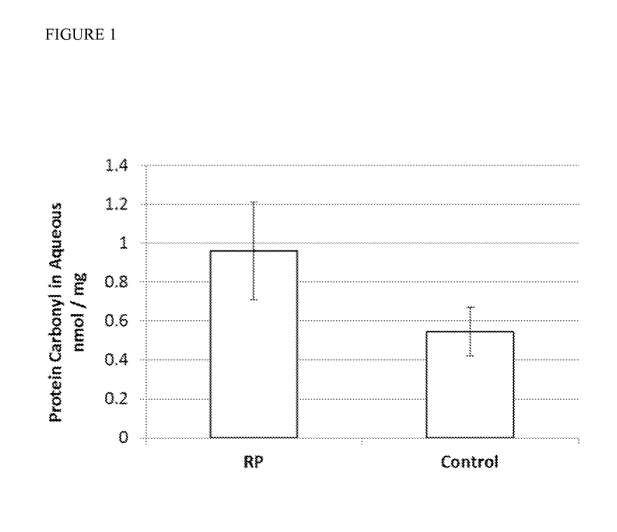

Aqueous humor samples were obtained from 9 patients with RP and 9 control patients who had a macular pucker or a macular hole. The total protein content of each sample was measured using the BioRad protein assay kit. Protein carbonyl content was measured in each sample using OxiSelect Protein Carbonyl ELISA kit (Cell Biolabs, Inc. San Diego, Calif.) using the manufacturer's instructions. Briefly, 50 μl of each sample or protein carbonyl standard was added to a well of a 96-well plate and incubated at 4° C. overnight. After washing, 100 μl of DNPH was added to each well and incubated for 45 minutes at room temperature. After 3 washes, blocking solution was applied to each well, and the plate was incubated for 1 hour at room temperature. Primary antibody was added and after incubation at room temperature for 1 hour, wells were washed three times. Secondary antibody was added and the plate was incubated for 1 hour at room temperature. After 3 washes, 100 μl of substrate was added to each well, the plate was incubated at room temperature for 25 minutes and then the reaction was stopped by adding 100 μl of stop solution. Absorbance at 450 nm was read on a plate reader. The readings from the standards were used to generate a standard curve which was used to determine the protein carbonyl content (nmol) per mg total protein of each sample. As shown in FIG. 1, each bar represents the mean (±standard deviation) protein carbonyl content per mg protein and statistical comparison was made by Student's unpaired t-test.

### Example 2

Comparison of the ratio of reduced glutathione to oxidized glutathione (GSH/GSSG) in aqueous samples from patients with retinitis pigmentosa (RP) and controls.

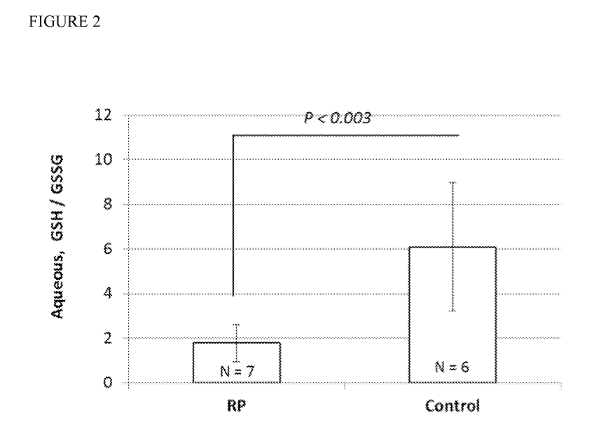

Aqueous humor samples were obtained from 7 patients with RP and 6 control patients who had a macular pucker or a macular hole. The GSH/GSSH ratio was measured by the methods of Queval (Analytical Biochemistry 363:58-69, 2007) and Owen (Analytical Biochemistry 106:207-212, 1980) by separate measurement of GSH and GSSG. For measurement of GSH, 50 μl of each aqueous humor sample, a GHS standard (ranging from 3 to 320 pmol), or blank phosphate-buffered solution buffer (PBS) were added to a well of a 96-well plate. After addition of 100 μl of a mixture of 2-nitrobenoic acid, NADPH, PBS and GSH reductase, plates were incubated at room temperature for 2 minutes and then absorbance at 405 nm was read on a plate reader. The absorbance values of the standards were plotted to generate a standard curve which was used to calculate the GSH level of each sample. For measurement of GSSG, 50 μl of each aqueous humor sample, a GSSG standard (ranging from 3 to 320 pmol), or blank PBS buffer were added to a well of a 96-well plate. A mixture of 2 μl of 2-vinylpyridine and 6 μl of triethanolamine was added to one well. After addition of 100 μl of a mixture of 2-nitrobenoic acid, NADPH, PBS and GSH reductase was added to each well, the plate was incubated at room temperature for 2 minutes, and then absorbance at 405 nm was read on a plate reader. The level of GSSG in each sample was calculated by comparison to the standard curve. As shown in FIG. 2, the bars represent the mean (±standard deviation) GSH/GSSH ratio and statistical comparison was made by unpaired t-test.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1.-3. (canceled)

4. A method for treating a subject having Retinitis Pigmentosa comprising:
a) measuring one or more of the following: cupric reducing antioxidant capacity, GSH/GSSG ratio, carbonyl adduct level in the biological sample from the subject and from a control biological sample;
b) comparing the one or more measurements of a) in the sample from the subject to the control sample;
c) quantifying or staging the severity of disease in the subject as progressing in stage, or increasing in severity, if the levels of cupric reducing antioxidant capacity and/or GSH/GSSG ratio in the sample from the subject is reduced relative to the control sample, and/or if the levels of carbonyl adduct level is elevated in the sample from the subject relative to the control sample; and
d) selecting a course of treatment for the disease in the subject which is based on the stage or severity of disease indicated in c).

5. The method of claim 4, wherein the sample is from the aqueous humor of the eye of the subject.

6. The method of claim 4, wherein the sample is from the blood or plasma of the subject.

7. A method for monitoring the treatment of a subject having Retinitis Pigmentosa comprising:
a) measuring one or more of the following: cupric reducing antioxidant capacity, GSH/GSSG ratio, carbonyl adduct level in the biological sample from the subject and from a control biological sample;
b) comparing the one or more measurements of a) in the sample from the subject to the control sample;
c) determining the stage or the severity of disease in the subject, wherein if the levels of cupric reducing antioxidant capacity and/or GSH/GSSG ratio is reduced in the sample from the subject relative to the control sample, and/or if the levels of carbonyl adduct level is elevated in the sample from the subject relative to the control sample then the disease is identified as progressing in stage, or increasing in severity, or if the levels of cupric reducing antioxidant capacity and/or GSH/GSSG ratio is elevated in the sample from the subject relative to the control sample and/or if the levels of carbonyl adduct level is reduced in the sample from the subject relative to the control sample then the disease is identified as reducing in stage, or decreasing in severity;
d) selecting a course of treatment for the disease in the subject which is based on the stage or severity of disease indicated in c); and
e) after administration of the course of treatment to the subject, repeating steps a)-d) one or more times.

8. The method of claim 7, wherein the sample is from the aqueous humor of the eye of the subject.

9. The method of claim 7, wherein the sample is from the blood or plasma of the subject.
